(19) United States
(12) Patent Application Publication
(10) Pub. No.: US 2022/0046930 A1
(43) Pub. Date: Feb. 17, 2022

(54) METHOD FOR INCREASING STORAGE STABILITY OF FUNGAL SPORES

(71) Applicant: Bayer Cropscience Biologics GMBH, Wismar (DE)

(72) Inventors: Bernhardt Michael STEINWENDER, Wismar (DE); Daniel LANVER, Lübow (DE); Fred NEUMANN, Wendisch Rambow (DE); Martina WUTTKE, Leverkusen (DE)

(73) Assignee: Bayer Cropscience Biologics GMBH, Wismar (DE)

(21) Appl. No.: 17/276,754

(22) PCT Filed: Sep. 16, 2019
(86) PCT No.: PCT/EP2019/074714

§ 371 Date: Mar. 16, 2021

(30) Foreign Application Priority Data
Sep. 19, 2018 (EP) 18195351.4

(51) Int. Cl. A01N 63/30

(52) U.S. Cl. CPC A01N 63/30; A01P 3/00

(57) ABSTRACT

The present invention relates to a method of producing fungal spores with prolonged storage stability or of prolonging the storage stability of fungal spores comprising subjecting fungal spores to a drying procedure to result in a residual moisture content of 12% or less and a composition comprising fungal spores obtained by said method.

Biological control agents become more and more important in the area of plant protection, be it for combatting various fungal or insect pests or for improving plant health. Although also viruses are available which can be used as biological control agents, mainly those based on bacteria and fungi are used in this area so far. The most prominent form of biological control agents based on fungi are their asexual spores called conidia as well as blastospores, but also other fungal propagules may be promising agents, such as (micro)sclerotia, ascospores, basidiospores, chlamydospores or hyphal fragments.

Unlike many spores based on bacteria, such as Bacillus spores, many fungal spores are less robust and it has proven to be difficult to provide fungal spores in a form which meets the needs of commercial products, in particular acceptable storage stability at certain temperatures, if possible usually at room temperature. Whereas this problem in the past has been addressed mainly by developing improved formulations individually per fungal species, there is still the need to provide for a general method of improving storage stability of fungal spores which does not need extensive experimentation in order to find a suitable formulation per species.

Different views exist about the influence of moisture content on storage stability of fungal spores. Moore et al. (Biocontrol Science and Technology 1996, 6: 51- 61) describe a moisture content of 4 to 5% as suitable for storage of M. flavoviridae spores for 3 months using oil or dry storage. Recently, a combined approach of microencapsulation and drying of fungal spores was used to increase storage stability of an M. brunneum strain (Przyklenk et al., 2017, Journal of Microencapsulation 34 (5): 498-512). However, the two-step drying method using a laminar flow and a desiccator resulted in a viability of spores of only 50% after three months and 1% after 6 months. Notably, no residual water content is reported and high losses upon drying were observed. Desiccation has frequently been used in the past to obtain dried fungal spores. Daoust et al. Journal of Invertebrate Pathology 1983, 41: 151-160) achieved storage stability of M. anisopliae conidia of 35% after 12 months using this method. Kim et al. (Mycobiology 2014, 42(1): 59-65) report drying of Beauveria bassiana using air drying for up to 140 h, however without long-term experiments. Horaczek and Viernstein (Biological Control 2004, 31: 65-71) use three different drying methods in an attempt to stabilize fungal spores.

However, there is still a need to provide fungal spores with enhanced storage stability to a commercial level. This technical problem has at least partially been solved in the present invention.

Accordingly, in a first aspect, the present invention relates to a method of producing fungal spores with prolonged storage stability or of prolonging the storage stability of fungal spores comprising subjecting fungal spores to a drying procedure to result in a residual moisture content of less than 12%.

Fungal spores include sexually (e.g. oospores, zygospores or ascospores) and asexually (e. g. conidia and chlamydospores, but also uredospores, teleutospores and ustospores) formed spores. Preferably the spores are conidia.

Storage stability denotes the ability of fungal spores to germinate after a given time. In particular, storage stability at a commercial level means a germination rate of fungal spores of at least 80% after 3 months at room temperature, preferably after 6 months.

Prolonged storage stability in connection with the present invention means that fungal spores can be stored for at least 6 months with a germination rate of at least 80%. Preferably, fungal spores can be stored for even 8 or even 10 or even 12 months with this germination rate. As evident from the enclosed examples, fungal spores, such as those of Metarhizium brunneum, more particularly strain F52, may most preferably be stored for even 12 months with a germination rate of at least 80%. Whereas it is preferred that storage stability is present at room temperature, it will at least be present at lower temperature. Lower temperature may be starting with 4° C., preferably 10 ° C., more preferably 15° C.

It was found in connection with the present invention that drying fungal spores below a moisture content of 12% serves for enhancing storage stability of the fungal spores. Generally, it is believed that storage stability is the further increased the lower the moisture content is. In some cases, it may be that moisture content should not be reduced below a certain threshold.

Drying of spores may be effected using methods known in the art, in particular vacuum drying, spray drying, freeze drying or desiccation.

In connection with the present invention, the fungal spores are preferably dried using vacuum drying. Vacuum drying is a process in which materials are dried in a reduced pressure environment, which lowers the heat needed for rapid drying. Vacuum dryers offer low temperature drying of thermolabile/-sensitive materials. According to the present invention, during the vacuum drying process the moisture content of fungal spores is lowered by creating a vacuum or at least lowering the atmospheric pressure the spores are exposed to. The principle behind this technique it is to expose a substance, such as fungal spores, to low(er) pressure and a (slightly) higher temperature. As the boiling point decreases with the surrounding pressure, a lower temperature is needed to dry more efficiently (faster) while the material is not exposed to heat stress. Any kind of vacuum dryer may be used in the present invention. It is, however, preferred that plate/table dryers and/or conical dryers are used.

In the course of the present invention, it was surprisingly found that vacuum drying when used for reducing the moisture content of fungal spores even further increased the storage stability of said spores. Whereas it was commonly thought that vacuum drying could be too harsh a method because the loss of water would be too quick and the low pressure might stress/rupture/degenerate viable spores, it was now found that drying fungal spores to a residual moisture content of below 12% using vacuum drying results in more storage stable spores, i.e. spores with an enhanced shelf life.

Residual moisture content or (residual) water content in connection with this application relates to the content of water present in the spores. It can e.g. be measured using a drying scale. This method is also known as “loss on drying” method. An exemplary setup is as follows: a 2-3 g sample of spore powder is put in the drying scale and the initial weight is measured (starting point). After that the drying scale heats up the sample and measures the decreasing weight (as water evaporates) over time. When the weight remains constant, the measurement is stopped and the loss of weight is calculated and expressed in % of the initial weight and corresponds to the residual moisture. The optimal residual moisture content for fungal spores may vary depending on the type of spores and the fungal species. In connection with the present invention, the residual moisture content of fungal spores is less than 12%. Generally, it may range between 1% and below 12%, preferably between 1% and below 8%, such as at 2, 3, 4, 5, 6 or 7% or any value in between, preferably between 1.5% and 6%, such as 2% or lower, 3% or lower, 4% or lower, 5% or lower or 6% or lower. For the sake of clarity, if not indicated otherwise, % as used in the present application refers to wt. %.

In the present method, vacuum drying is preferably effected for up to 16 hours. Accordingly, the drying period is generally chosen for periods of 16 hours or less, e.g. for between 30 minutes and 14 hours, preferably 1 to 12 hours, or any value in between, such as 2, 3, 4, 5, 6, 7, 8, 9, 10 or 11 hours. During the drying process, the spores may be agitated or left as is. The decision of whether to agitate the spores depends, inter alia, on the thickness of spore layer(s).

In the course of the present invention it has been found that, unlike described in the literature, comparably short drying processes are suitable for increasing storage stability of fungal spores, in particular conidia.

Depending on the fungal species and the types of spores as well as the initial moisture content, the length of the drying period may vary.

In one preferred embodiment, said vacuum drying is effected at a temperature of up to 40° C., preferably at up to 30° C., such as between 15 and 30° C. or 20 and 30° C.

In another preferred embodiment, said vacuum drying is effected at a pressure of between 50 and 0.5 mbar, preferably between 10 and 1 mbar.

Temperatures and pressures to be applied in the (vacuum) drying process depend on the fungal spore and will need to be adapted which is readily within the capabilities of the skilled person.

In a preferred embodiment, the fungal spores are conidia. Conidia are a kind of spores formed by fungi. Conidia are asexually formed and include but are not limited to aleurispores, anellospores, arthrospores, phialospores and pynidiospores. As opposed to e.g. bacterial spores, conidia are not intended to survive very harsh environmental conditions. In some embodiments, the conidia are hydrophobic. In others, the conidia are hydrophilic.

In another embodiment, the fungal spores are chlamydospores.

In one embodiment, the fungal spores are sexually formed. Sexually formed spores which can be used in the present invention include oospores, zygospores or ascospores.

The fungal microorganism the spores of which are used in the present invention is cultivated according to methods known in the art on an appropriate substrate, e. g. by submerged fermentation or solid-state fermentation, e. g. using a device disclosed in WO2005/012478 or WO1999/057239. It is preferred that the fungal spores are produced using solid-state fermentation as it has been found that the combination of producing the preferred spores, i.e. conidia, using solid-state fermentation followed by drying the spores, optionally before or after separation of the spores from the fermentation substrate, are a suitable basis for practicing the present invention. Accordingly, in one embodiment, the present method further comprises culturing a fungal microorganism using solid-state fermentation and harvesting the fungal spores produced. Spore material used for drying can be spore powder, spore suspension in compatible liquid such as water, slurry or paste.

The studies leading to the present invention were done under usual oxygen/air exposure. Accordingly, the observed storage stability is present even without oxygen depletion which is believed to further contribute to storage stability.

The fungal spores obtained using the method of the present invention may be applied in a suitable formulation. Preferably, such formulation provides for enhanced storage stability of the fungal spores resulting in even further enhanced storage stability.

Said fungal spores may originate from any fungus exerting a positive effect on plants such as a plant protective or plant growth promoting effect. Accordingly, said fungus may be an entomopathogenic fungus, a nematophagous fungus, a plant growth promoting fungus, a fungus active against plant pathogens such as bacteria or fungal plant pathogens, or a fungus with herbicidal action.

NRRL is the abbreviation for the Agricultural Research Service Culture Collection, an international depositary authority for the purposes of deposing microorganism strains under the Budapest treaty on the international recognition of the deposit of microorganisms for the purposes of patent procedure, having the address National Center for Agricultural Utilization Research, Agricultural Research service, U.S. Department of Agriculture, 1815 North university Street, Peroira, Ill. 61604 USA.

ATCC is the abbreviation for the American Type Culture Collection, an international depositary authority for the purposes of deposing microorganism strains under the Budapest treaty on the international recognition of the deposit of microorganisms for the purposes of patent procedure, having the address ATCC Patent Depository, 10801 University Blvd., Manassas, Va. 10110 USA.

If the fungal spores or the fungus growing from said fungal spores have a fungicidal effect, it may be selected from

B2.1 Coniothyrium minitans, in particular strain CON/M/91-8 (Accession No. DSM-9660; e.g. Contans® from Bayer CropScience Biologics GmbH); B2.2 Metschnikowia fructicola, in particular strain NRRL Y-30752; B2.3 Microsphaeropsis ochrace, in particular strain P130A (ATCC deposit 74412); B2.4 Muscodor albus, in particular strain QST 20799 (Accession No. NRRL 30547); B2.5 Trichoderma harzianum rifai, in particular strain KRL-AG2 (also known as strain T-22, ATCC 208479, e.g. PLANTSHIELD T-22G, Rootshield®, and TurfShield from BioWorks, US) and strain T39 (e.g. Trichodex® from Makhteshim, US); B2.6 Arthrobotrys dactyloides; B2.7 Arthrobotrys oligospora; B2.8 Arthrobotrys superba; B2.9 Aspergillus flavus, in particular strain NRRL 21882 (e.g. Afla-Guard® from Syngenta) or strain AF36 (e.g. AF36 from Arizona Cotton Research and Protection Council, US); B2.10 Gliocladium roseum (also known as Clonostachys rosea f. rosea), in particular strain 321U from Adjuvants Plus, strain ACM941 as disclosed in Xue (Efficacy of Clonostachys rosea strain ACM941 and fungicide seed treatments for controlling the root tot complex of field pea, Can Jour Plant Sci 83(3): 519-524), strain IK726 (Jensen D F, et al. Development of a biocontrol agent for plant disease control with special emphasis on the near commercial fungal antagonist Clonostachys rosea strain ‘IK726’; Australas Plant Pathol. 2007; 36: 95-101), strain 88-710 (WO2007/107000), strain CR7 (WO2015/035504) or strains CRrO, CRM and CRr2 disclosed in WO2017109802; B2.11 Phlebiopsis (or Phlebia or Peniophora) gigantea, in particular strain VRA 1835 (ATCC 90304), strain VRA 1984 (DSM16201), strain VRA 1985 (DSM16202), strain VRA 1986 (DSM16203), strain FOC PG B20/5 (IMI390096), strain FOC PG SP log6 (IMI390097), strain FOC PG SP log5 (IMI390098), strain FOC PG BU3 (IMI390099), strain FOC PG BU4 (IMI390100), strain FOC PG 410.3 (IMI390101), strain FOC PG 97/1062/116/1.1 (IMI390102), strain FOC PG B22/SP1287/3.1 (IMI390103), strain FOC PG SH1 (IMI390104) and/or strain FOC PG B22/SP1190/3.2 (IMI390105) (Phlebiopsis products are e.g. Rotstop® from Verdera and FIN, PG-Agromaster®, PG-Fungler®, PG-IBL®, PG-Poszwald® and Rotex® from e-nema, DE); B2.12 Pythium oligandrum, in particular strain DV74 or M1 (ATCC 38472; e.g. Polyversum from Bioprepraty, CZ); B2.13 Scleroderma citrinum; B2.14 Talaromyces flavus, in particular strain V117b; B2.15 Trichoderma asperellum, in particular strain ICC 012 from Isagro or strain SKT-1 (e.g. ECO-HOPE® from Kumiai Chemical Industry), strain T34 (e.g. T34 Biocontrol by Biocontrol Technologies S.L., ES); B2.16 Trichoderma atroviride, in particular strain CNCM 1-1237 (e.g. Esquive® WP from Agrauxine, FR), strain SC1 described in International Application No. PCT/IT2008/000196), strain 77B (T77 from Andermatt Biocontrol), strain no. V08/002387, strain NMI no. V08/002388, strain NMI no. V08/002389, strain NMI no. V08/002390, strain LC52 (e.g. Sentinel from Agrimm Technologies Limited), strain LUI32 (e.g. Tenet by Agrimm Technologies Limited), strain ATCC 20476 (IMI 206040), strain T11 (IM1352941/ CECT20498), strain SKT-1 (FERM P-16510), strain SKT-2 (FERM P-16511), strain SKT-3 (FERM P-17021); B2.17 Trichoderma harmatum; B2.18 Trichoderma harzianum, in particular, strain KD, strain ITEM 908 (e.g. Trianum-P from Koppert), strain TH35 (e.g. Root-Pro by Mycontrol), strain DB 103 (e.g. T-Gro 7456 by Dagutat Biolab); B2.19 Trichoderma virens (also known as Gliocladium virens), in particular strain GL-21 (e.g. SoilGard by Certis, US); B2.20 Trichoderma viride, in particular strain TV1, strain B35 (Pietr et al., 1993, Zesz. Nauk. A R w Szczecinie 161: 125-137); B2.21 Ampelomyces quisqualis, in particular strain AQ 10 (e.g. AQ 10® by CBC Europe, Italy); B2.22 Arkansas fungus 18, ARF; B2.23 Aureobasidium pullulans, in particular blastospores of strain DSM14940, blastospores of strain DSM 14941 or mixtures of blastospores of strains DSM14940 and DSM 14941 (e.g. Botector® by bio-ferm, CH); B2.24 Chaetomium cupreum (e.g. BIOKUPRUM TM by AgriLife); B2.25 Chaetomium globosum (e.g. Rivadiom by Rivale); B2.26 Cladosporium cladosporioides, in particular strain H39 (by Stichting Dienst Landbouwkundig Onderzoek); B2.27 Dactylaria candida; B2.28 Dilophosphora alopecuri (e.g. Twist Fungus); B2.29 Fusarium oxysporum, in particular strain Fo47 (e.g. Fusaclean by Natural Plant Protection); B2.30 Gliocladium catenulatum (Synonym: Clonostachys rosea f. catenulate), in particular strain J1446 (e.g. Prestop ® by Lallemand); B2.31 Lecanicillium lecanii (formerly known as Verticillium lecanii), in particular conidia of strain KV01 (e.g. Vertalec® by Koppert/Arysta); B2.32 Penicillium vermiculatum; B2.33 Trichoderma gamsii (formerly T. viride), in particular strain ICC080 (IMI CC 392151 CABI, e.g. BioDerma by AGROBIOSOL DE MEXICO, S.A. DE C.V.); B2.34 Trichoderma polysporum, in particular strain IMI 206039 (e.g. Binab TF WP by BINAB Bio-Innovation AB, Sweden); B2.35 Trichoderma stromaticum (e.g. Tricovab by Ceplac, Brazil); B2.36 Tsukamurella paurometabola, in particular strain C-924 (e.g. HeberNem®); B2.37 Ulocladium oudemansii, in particular strain HRU3 (e.g. Botry-Zen® by Botry-Zen Ltd, NZ); B2.38 Verticillium albo-atrum (formerly V. dahliae), in particular strain WCS850 (CBS 276.92; e.g. Dutch Trig by Tree Care Innovations); B2.39 Muscodor roseus, in particular strain A3-5 (Accession No. NRRL 30548); B2.40 Verticillium chlamydosporium; B2.41 mixtures of Trichoderma asperellum strain ICC 012 and Trichoderma gamsii strain ICC 080 (product known as e.g. BIO-TAM™ from Bayer CropScience LP, US), B2.42 Simplicillium lanosoniveum and B2.43 Trichoderma fertile (e.g. product TrichoPlus from BASF).

In a preferred embodiment, the fungal spores from the fungus having fungicidal activity is selected from Coniothyrium minitans, in particular strain CON/M/91-8 (Accession No. DSM-9660) (available as Contans® from Prophyta, Del.); Microsphaeropsis ochracea strain P130A (ATCC 74412); Aspergillus flavus, strain NRRL 21882 (available as Afla-Guard® from Syngenta) and strain AF36 (available as AF36 from Arizona Cotton Research and Protection Council, US); Gliocladium roseum, strain 321U from Adjuvants Plus; Talaromyces flavus, strain VII7b; Ampelomyces quisqualis, in particular strain AQ 10 (available as AQ 10® by IntrachemBio Italia); Gliocladium catenulatum (Synonym: Clonostachys rosea f. catenulate), in particular strain J1446 (e.g. Prestop ® by Verdera Oy), strain IK726, strain 88-710 (WO2007/107000), strain CR7 (WO2015/035504), Trichoderma asperellum, in particular strain ICC 012 from Isagro or strain SKT-1 (e.g. ECO-HOPE® from Kumiai Chemical Industry), strain T34 (e.g. T34 Biocontrol by Biocontrol Technologies S.L., ES); Trichoderma viride, in particular strain TV1(e.g. Trianum-P by Koppert) or strain B35 (Pietr et al., 1993, Zesz. Nauk. A R w Szczecinie 161: 125-137), Trichoderma atroviride, in particular strain CNCM 1-1237 (e.g. Esquive® WP from Agrauxine, FR) or strain SC1 described in International Application No. PCT/IT2008/000196), strain 77B (T77 from Andermatt Biocontrol), strain no. V08/002387, strain NMI no. V08/002388, strain NMI no. V08/002389, strain NMI no. V08/002390, strain LC52 (e.g.

Sentinel from Agrimm Technologies Limited), strain LUI32 (e.g. Tenet by Agrimm Technologies Limited), strain ATCC 20476 (IMI 206040), strain T11 (IMI352941/ CECT20498), strain SKT-1 (FERM P-16510), strain SKT-2 (FERM P-16511), strain SKT-3 (FERM P-17021) and Cladosporium cladosporioides, e. g. strain H39 (by Stichting Dienst Landbouwkundig Onderzoek).

In an even more preferred embodiment, the fungal spores are from a fungus having fungicidal activity selected from Coniothyrium minitans, in particular strain CON/M/91-8 (Accession No. DSM-9660) (available as Contans ® from Prophyta, DE); Gliocladium catenulatum (Synonym: Clonostachys rosea f. catenulate), in particular strain J1446 (e.g. Prestop ® by Lallemand), strain IK726, strain 88-710 (WO2007/107000), strain CR7 (WO2015/035504); Trichoderma viride, in particular strain TV1 (e.g. Trianum-P by Koppert), strain B35 (Pietr et al., 1993, Zesz. Nauk. A R w Szczecinie 161: 125-137) and Trichoderma atroviride, in particular strain CNCM 1-1237 (e.g. Esquive® WP from Agrauxine, FR), strain SC1 described in International Application No. PCT/IT2008/000196), strain 77B (T77 from Andermatt Biocontrol).

It is most preferred that the fungal spores from a fungus having fungicidal activity are from Coniothyrium minitans, in particular strain CON/M/91-8, Trichoderma viride strain B35 and Trichoderma atroviride, in particular strain CNCM 1-1237.

In one especially preferred embodiment, the fungal spores are from Coniothyrium minitans, in particular strain CON/M/91-8. In another especially preferred embodiment, the fungal spores are from Trichoderma viride strain B35. In yet another especially preferred embodiment, the fungal spores are from Trichoderma atroviride strain CNCM 1-1237. It is even more preferred that in these embodiments, the spores are produced using solid-state fermentation. In all embodiments described in this paragraph, it is preferred that the drying process is vacuum drying as described elsewhere in this application.

If the fungal spores or the fungus growing from said fungal spores have an insecticidal effect (entomopathogenic fungus), said fungus may be selected from C2.1 Muscodor albus, in particular strain QST 20799 (Accession No. NRRL 30547); C2.2 Muscodor roseus in particular strain A3-5 (Accession No. NRRL 30548); C2.3 Beauveria bassiana, in particular strain ATCC 74040 (e.g. Naturalis® from CBC Europe, Italy; Contego BB from Biological Solutions Ltd.; Racer from AgriLife); strain GHA (Accession No. ATCC74250; e.g. BotaniGuard Es and Mycotrol-O from Laverlam International Corporation); strain ATP02 (Accession No. DSM 24665); strain PPRI 5339 (e.g. BroadBand™ from BASF); strain PPRI 7315, strain R444 (e.g. Bb-Protec from Andermatt Biocontrol), strains IL197, IL12, IL236, IL10, IL131, IL116 (all referenced in Jaronski, 2007. Use of Entomopathogenic Fungi in Biological Pest Management, 2007: ISBN: 978-81-308-0192-6), strain Bv025 (see e.g. Garcia et al. 2006. Manejo Integrado de Plagas y Agroecología (Costa Rica) No. 77); strain BaGPK; strain ICPE 279, strain CG 716 (e.g. BoveMax® from Novozymes); C2.4 Hirsutella citriformis; C2.5 Hirsutella thompsonii (e.g. Mycohit and ABTEC from Agro Bio-tech Research Centre, IN); C2.6 Lecanicillium lecanii (formerly known as Verticillium lecanii), in particular conidia of strain KV01 (e.g. Mycotal® and Vertalec® from Koppert/Arysta), strain DAOM198499 or strain DAOM216596; C2.9 Lecanicillium muscarium (formerly Verticillium lecanii), in particular strain VE 6/CABI(=IMI) 268317/CBS102071/ARSEF5128 (e.g. Mycotal from Koppert); C2.10 Metarhizium anisopliae var acridum, e.g. ARSEF324 from GreenGuard by BASF, US or isolate IMI 330189 (ARSEF7486; e.g. Green Muscle by Biological Control Products); C2.11 Metarhizium brunneum, e.g. strain Cb 15 (e.g. ATTRACAP® from BIOCARE); or strain F52 (DSM3884/ ATTC90448; e.g. BIO 1020 by Bayer CropScience and also e.g. Met52 by Novozymes); C2.12 Metarhizium anisopliae complex species/strains, e.g. strain ESALQ 1037 (e.g. from Metarril® SP Organic), strain E-9 (e.g. from Metarril® SP Organic), strain M206077, strain C4-B (NRRL 30905), strain ESC1, strain 15013-1 (NRRL 67073), strain 3213-1 (NRRL 67074), strain C20091, strain C20092, or strain ICIPE 78; C2.15 Metarhizium robertsii 23013-3 (NRRL 67075); C2.13 Nomuraea rileyi; C2.14 Paecilomyces fumosoroseus (new: Isaria fumosorosea), in particular strains Apopka 97 (available as PreFeRal from Certis, USA), Fe9901 (available as NoFly from Natural industries, USA), ARSEF 3581, ARSEF 3302, ARSEF 2679 (ARS Collection of Entomopathogenic Fungal Cultures, Ithaca, USA), IfB01 (China Center for Type Culture Collection CCTCC M2012400), ESALQ1296, ESALQ1364, ESALQ1409 (ESALQ: University of São Paulo (Piracicaba, S P, Brazil)), CG1228 (EMBRAPA Genetic Resources and Biotechnology (Brasília, D F, Brazil)), KCH J2 (Dymarska et al., 2017; PLoS one 12(10)): e0184885), HIB-19, HIB-23, HIB-29, HIB-30 (Gandarilla-Pacheco et al., 2018; Rev Argent Microbiol 50: 81-89), CHE-CNRCB 304, EH-511/3 (Flores-Villegas et al., 2016; Parasites & Vectors 2016 9:176 doi: 10.1186/s13071-016-1453-1), CHE-CNRCB 303, CHE-CNRCB 305, CHE-CNRCB 307 (Gallou et al., 2016; fungal biology 120 (2016) 414-423), EH-506/3, EH-503/3, EH-520/3, PFCAM, MBP, PSMB1 (National Center for Biololgical Control, Mexico; Castellanos-Moguel et al., 2013; Revista Mexicana De Micologia 38: 23-33, 2013), RCEF3304 (Meng et al., 2015; Genet Mol Biol. 2015 July-September; 38(3): 381-389), PF01-N10 (CCTCC No. M207088), CCM 8367 (Czech Collection of Microorganisms, Brno), SFP-198 (Kim et al., 2010; Wiley Online: DOI 10.1002/ps.2020), K3 (Yanagawa et al., 2015; J Chem Ecol. 2015; 41(12): 118-1126), CLO 55 (Ansari Ali et al., 2011; PLoS One. 2011; 6(1): e16108. DOI: 10.1371/journal.pone.0016108), IfTS01, IfTS02, IfTS07 (Dong et al. 2016/PLoS ONE 11(5): e0156087. doi:10.1371/journal.pone.0156087), P1 (Sun Agro Biotech Research Centre, India), If-02, If-2.3, If-03 (Farooq and Freed, 2016; DOI: 10.1016/j.bjm.2016.06.002), Ifr AsC (Meyer et al., 2008; J. Invertebr. Pathol. 99:96-102. 10.1016/j.jip.2008.03.007), PC-013 (DSMZ 26931), P43A, PCC (Carrillo-Perez et al., 2012; DOI 10.1007/s11274-012-1184-1), Pf04, Pf59, Pf109 (KimJun et al., 2013; Mycobiology 2013 Dec; 41(4): 221-224), FG340 (Han et al., 2014; DOI: 10.5941/MYCO.2014.42.4.385), Pfr1, Pfr8, Pfr9, Pfr10, Pfr11, Pfr12 (Angel-Sahagún et al., 2005; Journal of Insect Science), Ifr531 (Daniel and Wyss, 2009; DOI 10.1111/j.1439-0418.2009.01410.x), IF-1106 (Insect Ecology and Biocontrol Laboratory, Shanxi Agricultural University), I9602, I7284 (Hussain et al. 2016, DOI: 10.3390/ijms17091518), I03011 (Patent U.S. Pat. No. 4,618,578), CNRCB1 (Centro Nacional de Referencia de Control Biologico (CNRCB), Colima, Mexico), SCAU-IFCF01 (Nian et al., 2015; DOI: 10.1002/ps.3977), PF01-N4 (Engineering Research Center of Biological Control, SCAU, Guangzhou, P. R. China) Pfr-612 (Institute of Biotechnology (IB-FCB-UANL), Mexico), Pf-Tim, Pf-Tiz, Pf-Hal, Pf-Tic (Chan-Cupul et al. 2013, DOI: 10.5897/AJMR12.493); C2.15 Aschersonia aleyrodis; C2.16 Beauveria brongniartii (e.g. Beaupro from Andermatt Biocontrol AG); C2.17 Conidiobolus obscurus; C2.18 Entomophthora virulenta (e.g. Vektor from Ecomic); C2.19 Lagenidium giganteum; C2.20 Metarhizium flavoviride; C2.21 Mucor haemelis (e.g. BioAvard from Indore Biotech Inputs & Research); C2.22 Pandora delphacis; C2.23 Sporothrix insectorum (e.g. Sporothrix Es from Biocerto, BR); C2.24 Zoophtora radicans.

In a preferred embodiment, said fungal spores originate from a fungal species selected from the group consisting of Isaria fumosorosea, Penicillium frequentans, Cladosporium cladosporioides, Cladosporium delicatum, Metarhizium spp., Beauveria bassiana, Beauveria brogniartii, Lecanicillium spp., Clonostachys rosea, Nomuraea rileyi, Trichoderma spp., Penicillium bilaii, Coniothyrium minitans and Purpureocillium lilacinum.

In another preferred embodiment, said fungal spores originate from an entomopathogenic fungus, i.e. have insecticidal activity. These include the above-listed species such as Isaria fumosorosea, Metarhizium spp., Beauveria bassiana, Beauveria brogniartii, Lecanicillium spp. and Nomuraea rileyi.

In a more preferred embodiment, fungal strains having an insecticidal effect may be selected from C2.3 Beauveria bassiana, in particular strain ATCC 74040; strain GHA (Accession No. ATCC74250); strain ATP02 (Accession No. DSM 24665); strain PPRI 5339; strain PPRI 7315, strain R444, strains IL197, IL12, IL236, IL10, IL131, IL116; strain BaGPK; strain ICPE 279, strain CG 716; C2.6 Lecanicillium lecanii (formerly known as Verticillium lecanii), in particular conidia of strain KV01, strain DAOM198499 or strain DAOM216596; C2.9 Lecanicillium muscarium (formerly Verticillium lecanii), in particular strain VE 6/CABI(=IMI) 268317/CBS102071/ARSEF5128; C2.10 Metarhizium acridum, e.g. ARSEF324 or isolate IMI 330189 (ARSEF7486); C2.11 Metarhizium brunneum, e.g. strain Cb 15 or strain F52 (DSM3884/ATCC 90448); C2.12 Metarhizium anisopliae complex species/strains, e.g. strain ESALQ 1037, strain E-9, strain M206077, strain C4-B (NRRL 30905), strain ESC1, strain 15013-1 (NRRL 67073), strain 3213-1 (NRRL 67074), strain C20091, strain C20092, or strain ICIPE 78; C2.14 Paecilomyces fumosoroseus (new: Isaria fumosorosea), in particular strains Apopka 97, Fe9901, ARSEF 3581, ARSEF 3302, ARSEF 2679, IfB01 (China Center for Type Culture Collection CCTCC M2012400), ESALQ1296, ESALQ1364, ESALQ1409, CG1228, KCH J2, HIB-19, HIB-23, HIB-29, HIB-30, CHE-CNRCB 304, EH-511/3, CHE-CNRCB 303, CHE-CNRCB 305, CHE-CNRCB 307, EH-506/3, EH-503/3, EH-520/3, PFCAM, MBP, PSMB1, RCEF3304, PF01-N10 (CCTCC No. M207088), CCM 8367, SFP-198, K3, CLO 55, IfTS01, IfTS02, IfTS07, P1, If-02, If-2.3, If-03, Ifr AsC, PC-013 (DSMZ 26931), P43A, PCC, Pf04, Pf59, Pf109, FG340, Pfr1, Pfr8, Pfr9, Pfr10, Pfr11, Pfr12, Ifr531, IF-1106, 19602, 17284 , 103011 (Patent U.S. Pat. No. 4,618,578), CNRCB1, SCAU-IFCF01, PF01-N4, Pfr-612, Pf-Tim, Pf-Tiz, Pf-Hal and Pf-Tic.

It is even more preferred that the fungal strain is of the species Isaria fumosorosea. Preferred strains of Isaria fumosorosea are selected from the group consisting of Apopka 97, Fe9901, ARSEF 3581, ARSEF 3302, ARSEF 2679, IfB01 (China Center for Type Culture Collection CCTCC M2012400), ESALQ1296, ESALQ1364, ESALQ1409, CG1228, KCH J2, HIB-19, HIB-23, HIB-29, HIB-30, CHE-CNRCB 304, EH-511/3, CHE-CNRCB 303, CHE-CNRCB 305, CHE-CNRCB 307, EH-506/3, EH-503/3, EH-520/3, PFCAM, MBP, PSMB1, RCEF3304, PF01-N10 (CCTCC No. M207088), CCM 8367, SFP-198, K3, CLO 55, IfTS01, IfTS02, IfTS07, P1, If-02, If-2.3, If-03, Ifr AsC, PC-013 (DSMZ 26931), P43A, PCC, Pf04, Pf59, Pf109, FG340, Pfr1, Pfr8, Pfr9, Pfr10, Pfr11, Pfr12, Ifr531, IF-1106, 19602, 17284 , 103011 (Patent U.S. Pat. No. 4,618,578), CNRCB1, SCAU-IFCF01, PF01-N4, Pfr-612, Pf-Tim, Pf-Tiz, Pf-Hal, Pf-Tic. It is most preferred that said Isaria fumosorosea strain is selected from Apopka 97 and Fe9901. A particularly preferred strain is APOPKA97. For the species Isaria fumosorosea it is preferred that the spores are dried to a residual moisture content of between 5 and 10%, preferably between 6 and 8%.

Among entomopathogenic fungi, those of the genus Metarhizium spp. are especially preferred. The genus Metahrizium comprises several species some of which have recently been re-classified (for an overview, see Bischoff et al., 2009; Mycologia 101 (4): 512-530). Members of the genus Metarhizium comprise M. pingshaense, M. anisopliae, M. robertsii, M. brunneum (these four are also referred to as Metarhizium anisopliae complex), M. acridum, M. majus, M. guizouense, M. lepidiotae and M. globosum. Of these, M. anisopliae, M. robertsii, M. brunneum and M. acridum are even more preferred, whereas those of M. brunneum and M. acridum are most preferred.

Exemplary strains belonging to Metarhizium spp. which are also especially preferred are Metarhizium acridum ARSEF324 (product GreenGuard by BASF) or isolate IMI 330189 (ARSEF7486; e.g. Green Muscle by Biological Control Products); Metarhizium brunneum strain Cb 15 (e.g. ATTRACAP® from BIOCARE), or strain F52 (DSM3884/ATCC 90448; e.g. BIO 1020 by Bayer CropScience and also e.g. Met52 by Novozymes); Metarhizium anisopliae complex strains strain ESALQ 1037 or strain ESALQ E-9 (both from Metarril® WP Organic), strain M206077, strain C4-B (NRRL 30905), strain ESC1, strain 15013-1 (NRRL 67073), strain 3213-1 (NRRL 67074), strain C20091, strain C20092, or strain ICIPE 78. Most preferred are isolate F52 (a.k.a. Met52) which primarily infects beetle larvae and which was originally developed for control of Otiorhynchus sulcatus. and ARSEF324 which is commercially used in locust control. Commercial products based on the F52 isolate are subcultures of the individual isolate F52 and are represented in several culture collections including: Julius Kuhn-Institute for Biological Control (previously the BBA), Darmstadt, Germany: [as M.a. 43]; HRI, UK: [275-86 (acronyms V275 or KVL 275)]; KVL Denmark [KVL 99-112 (Ma 275 or V 275)]; Bayer, Germany [DSM 3884]; ATCC, USA [ATCC 90448]; USDA, Ithaca, USA [ARSEF 1095]. Granular and emulsifiable concentrate formulations based on this isolate have been developed by several companies and registered in the EU and North America (US and Canada) for use against black vine weevil in nursery ornamentals and soft fruit, other Coleoptera, western flower thrips in greenhouse ornamentals and chinch bugs in turf.

Beauveria bassiana is mass-produced and used to manage a wide variety of insect pests including whiteflies, thrips, aphids and weevils. Lecanicillium spp. is deployed against white flies, thrips and aphids. Metarhizium spp. is used against pests including beetles, locusts and other grasshoppers, Hemiptera, and spider mites. Isaria fumosorosea is effective e.g. against white flies, thrips and aphids.

Preferred strains of Beauveria bassiana include strain ATCC 74040; strain GHA (Accession No. ATCC74250); strain ATP02 (Accession No. DSM 24665); strain PPRI 5339; strain PPRI 7315, strains IL197, IL12, IL236, IL10, IL131, IL116, strain Bv025; strain BaGPK; strain ICPE 279, strain CG 716; ESALQPL63, ESALQ447 and ESALQ1432, CG1229 , IMI389521, NPP111B005, Bb-147.

It is most preferred that Beauveria bassiana strains include strain ATCC 74040 and strain GHA (Accession No. ATCC74250).

For the species Beauveria bassiana it is preferred that the spores are dried to a residual moisture content of between 2 and 6%, preferably between 3 and 5%, such as 4%.

In one especially preferred embodiment, the fungal spores are from Metarhizium brunneum strain F52. In another especially preferred embodiment, the fungal spores are from Paecilomyces fumosoroseus (new: Isaria fumosorosea) strain Apopka 97. In yet another especially preferred embodiment, the fungal spores are from Beauveria bassiana strain PPRI 5339. It is even more preferred that in these embodiment, the spores are produced using solid-state fermentation. In all embodiments desribed in this paragraph, it is preferred that the drying process is vacuum drying as described elsewhere in this application.

If the fungal spores or the fungus growing from said fungal spores have a nematicidal effect, it may be selected from

D2.1 Muscodor albus, in particular strain QST 20799 (Accession No. NRRL 30547); D2.2 Muscodor roseus, in particular strain A3-5 (Accession No. NRRL 30548); D2.3 Purpureocillium lilacinum (formerly known as Paecilomyces lilacinus), in particular P. lilacinum strain 251 (AGAL 89/030550; e.g. BioAct from Bayer CropScience Biologics GmbH), strain 580 (BIOSTAT® WP (ATCC No. 38740) by Laverlam), strain in the product BIO-NEMATON® (T.Stanes and Company Ltd.), strain in the product MYSIS® (Varsha Bioscience and Technology India Pvt Ltd.), strain in the product BIOICONEMA® (Nico Orgo Maures, India), strain in the product NEMAT® (Ballagro Agro Tecnologia Ltda, Brazil), and a strain in the product SPECTRUM PAE L® (Promotora Tecnica Industrial, S.A. DE C.V., Mexico); D2.4 Trichoderma koningii; D2.5 Harposporium anguillullae; D2.6 Hirsutella minnesotensis; D2.7 Monacrosporium cionopagum; D2.8 Monacrosporium psychrophilum; D2.9 Myrothecium verrucaria, in particular strain AARC-0255 (e.g. DiTera™ from Valent Biosciences); D2.10 Paecilomyces variotii, strain Q-09 (e.g. Nemaquim® from Quimia, MX); D2.11 Stagonospora phaseoli (e.g. from Syngenta); D2.12 Trichoderma lignorum, in particular strain TL-0601 (e.g. Mycotric from Futureco Bioscience, ES); D2.13 Fusarium solani, strain Fs5; D2.14 Hirsutella rhossiliensis; D2.15 Monacrosporium drechsleri; D2.16 Monacrosporium gephyropagum; D2.17 Nematoctonus geogenius; D2.18 Nematoctonus leiosporus; D2.19 Neocosmospora vasinfecta; D2.20 Paraglomus sp, in particular Paraglomus brasilianum; D2.21 Pochonia chlamydosporia (also known as Vercillium chlamydosporium), in particular var. catenulate (such as IMI SD 187; e.g. KlamiC from The National Center of Animal and Plant Health (CENSA), CU); D2.22 Stagonospora heteroderae; D2.23 Meristacrum asterospermum, D2.24 Duddingtonia flagrans.

In a more preferred embodiment, fungal strains with nematicidal effect are selected from Purpureocillium lilacinum, in particular spores of P. lilacinum strain 251 (AGAL 89/030550) (available as BioAct from Prophyta) and Duddingtonia flagrans. It is most preferred that the fungal strain with nematicidal activity is P. lilacinum strain 251. It is even more preferred that in this embodiment, the spores are produced using solid-state fermentation. Furthermore, it is preferred that the drying process is vacuum drying as described elsewhere in this application.

If the fungal spores or the fungus growing from said fungal spores support and/or promote and/or stimulate plant health and plant growth they may be selected from

E2.1 Talaromyces flavus, in particular strain V117b; E2.2 Trichoderma atroviride, in particular strain CNCM 1-1237 (e.g. Esquive® WP from Agrauxine, FR), strain SC1 described in International Application No. PCT/IT2008/000196), strain no. V08/002387, strain no. V08/002387, strain no. NMI No. V08/002388, strain no. NMI No. V08/002389, strain no. NMI No. V08/002390, strain LC52 (e.g. Sentinel from Agrimm Technologies Limited), strain kd (e.g. T-Gro from Andermatt Biocontrol), and/or strain LUI32 (e.g. Tenet from Agrimm Technologies Limited); E2.3 Trichoderma harzianum, in particular strain ITEM 908 or T-22 (e.g. Trianum-P from Koppert); E2.4 Myrothecium verrucaria, in particular strain AARC-0255 (e.g. DiTera™ from Valent Biosciences); E2.5 Penicillium bilaii, in particular strain ATCC 22348 and/or strain ATCC20851 (e.g. JumpStart® from Monsanto BioAg); E2.6 Pythium oligandrum, in particular strains DV74 or M1 (ATCC 38472; e.g. Polyversum from Bioprepraty, CZ); E2.7 Rhizopogon amylopogon (e.g. comprised in Myco-Sol from Helena Chemical Company); E2.8 Rhizopogon fulvigleba (e.g. comprised in Myco-Sol from Helena Chemical Company); E2.9 Trichoderma harzianum, in particular strain TSTh20, strain KD, product Eco-T from Plant Health Products, ZA or strain 1295-22; E2.10 Trichoderma koningii; E2.11 Glomus aggregatum; E2.12 Glomus clarum; E2.13 Glomus deserticola; E2.14 Glomus etunicatum; E2.15 Glomus intraradices; E2.16 Glomus monosporum; E2.17 Glomus mosseae; E2.18 Laccaria bicolor; E2.19 Rhizopogon luteolus; E2.20 Rhizopogon tinctorus; E2.21 Rhizopogon villosulus; E2.22 Scleroderma cepa; E2.23 Suillus granulatus; E2.24 Suillus punctatapies; E2.25 Trichoderma virens, in particular strain GL-21; and E2.26 Verticillium albo-atrum (formerly V. dahliae), in particular strain WCS850 (CBS 276.92; e.g. Dutch Trig from Tree Care Innovations), E2.27 Trichoderma viride, e.g. strain B35 (Pietr et al., 1993, Zesz. Nauk. A R w Szczecinie 161: 125-137) and E2.28 Purpureocillium lilacinum (previously known as Paecilomyces lilacinus) strain 251 (AGAL 89/030550; e.g. BioAct from Bayer CropScience Biologics GmbH).

In a more preferred embodiment, fungal strains having a beneficial effect on plant health and/or growth are selected from Talaromyces flavus, strain VII7b; Trichoderma harzianum strain KD or strain in product Eco-T from Plant Health Products, SZ; Penicillium bilaii strain ATCC 22348, and/or strain ATCC20851; Trichoderma virens strain GL-21, Trichoderma viride strain B35; Trichoderma atroviride strain CNCM I-1237, and Purpureocillium lilacinum (previously known as Paecilomyces lilacinus) strain 251 (AGAL 89/030550).

In an even more preferred embodiment, fungal strains having a beneficial effect on plant health and/or growth are selected from Penicillium bilaii, in particular strain ATCC 22348, Trichoderma viride strain B35, Trichoderma atroviride strain CNCM 1-1237 and Purpureocillium lilacinum (previously known as Paecilomyces lilacinus) strain 251 (AGAL 89/030550).

In an alternative particularly preferred embodiment, fungal strains having a beneficial effect on plant health and/or growth are selected from Trichoderma harzianum, in particular strain KD, strain ITEM 908 or strain T-22, Trichoderma virens, in particular strain GL-21, and Trichoderma viride, in particular strain B35 (Pietr et al., 1993, Zesz. Nauk. A R w Szczecinie 161: 125-137).

In one especially preferred embodiment, the fungal spores are from Penicillium bilaii, in particular strain ATCC 22348. In a nother especially preferred embodiment, the fungal spores are from Trichoderma viride strain B35. In yet another especially preferred embodiment, the fungal spores are from Trichoderma atroviride strain CNCM 1-1237. In another especially preferred embodiment, the fungal spores are from Purpureocillium lilacinum (previously known as Paecilomyces lilacinus) strain 251 (AGAL 89/030550). It is even more preferred that in these embodiment, the spores are produced using solid-state fermentation. In all embodiments desribed in this paragraph, it is preferred that the drying process is vacuum drying as described elsewhere in this application.

If the fungal spores or the fungus growing from said fungal spores have a herbicidal effect they may be selected from

F2.1 Phoma macrostroma, in particular strain 94-44B; F2.2 Sclerotinia minor, in particular strain IMI 344141 (e.g. Sarritor by Agrium Advanced Technologies); F2.3 Colletotrichum gloeosporioides, in particular strain ATCC 20358 (e.g. Collego (also known as LockDown) by Agricultural Research Initiatives); F2.4 Stagonospora atriplicis; or F2.5 Fusarium oxysporum, different strains of which are active against different plant species, e.g. the weed Striga hermonthica (Fusarium oxysproum formae specialis strigae).

The following examples illustrate the invention in a non-limiting fashion.

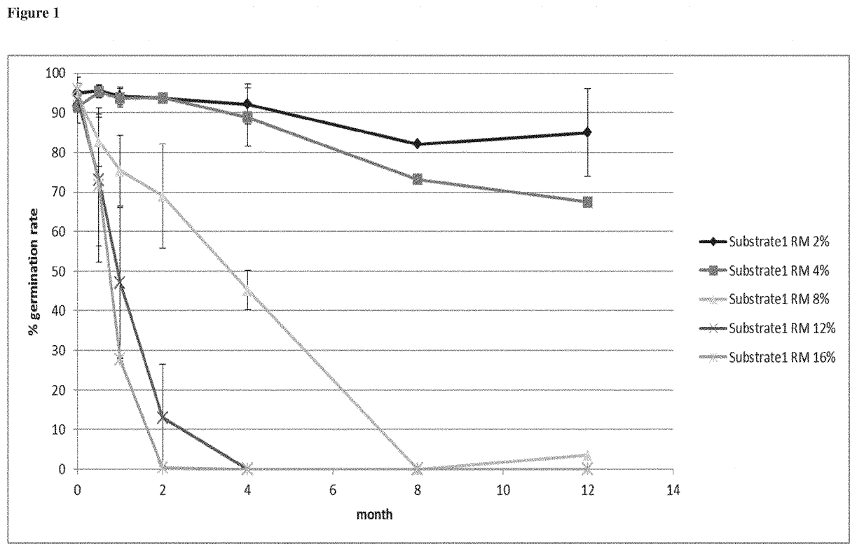

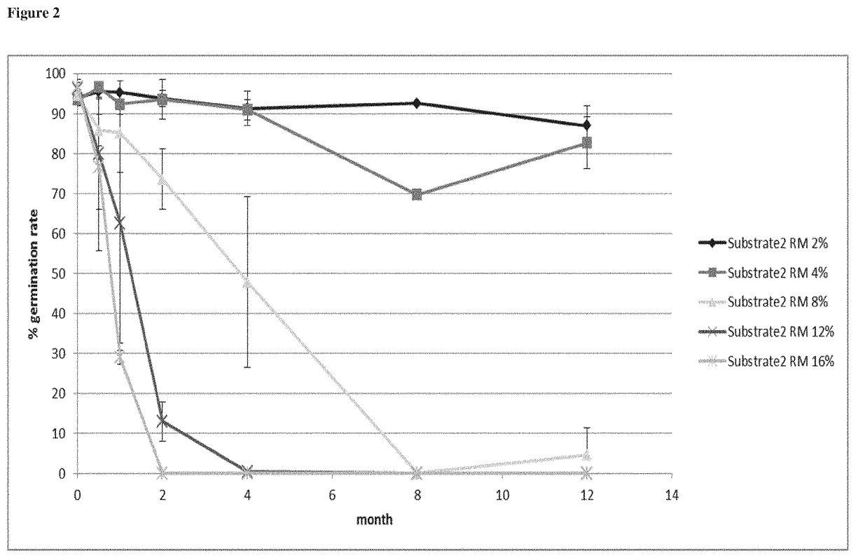

FIGS. 1 and 2: Comparison of germination rate of Metarhizium brunneum spores fermented on two different substrates with different residual moisture contents

### EXAMPLE 1

### Drying Procedure for Fungal Spores

The conidia powder (before drying usually around 50% residual moisture) is evenly distributed in thermal conducting trays, making sure the layer of the powder is not thicker than 2 cm. The trays then are placed in the respective vacuum-dryer. After that a vacuum is created in the drying chamber, enabling the residual moisture to evaporate from the powder. The moisture is pumped out of the drying chamber by the vacuum pump and the powder subsequently dried.

The drying trays are heated to a set temperature which is constantly adjusted to account for evaporative cooling. A temperature sensor in the powder shows the change in the temperature of the powder. If this temperature increases towards the temperature of the tray it indicates that at the given vacuum there is only little moisture left that will evaporate from the powder. Then a greater (bigger/stronger) vacuum can be created to enable more moisture to leave the powder or the powder reached the desired moisture level. The moisture level is determined by stopping the drying process, breaking the vacuum, taking a small, representative sample and analyzing it in an infrared moisture analyzer. If needed the drying process is then started again.

### EXAMPLE 2

### Determination of Germination Rate

To determine the germination rate of a spore powder sample at a point in time the vacuum sealed aluminum bags where the spores were stored at 25° C. were opened and a max. of 1 mg of spores was transferred to an Erlenmeyer flask (150 mL) and mixed into approx. 100 mL water comprising a low percentage of surfactant and agitated with a magnetic stirrer for approx. 30 minutes.

After that the suspension was diluted 10-100 fold and 100 μL each plated on 2 petri dishes with artificial media (Potatoe dextrose agar+Chloramphenicol+Benomyl). The Petri dishes were incubated for 40 h in darkness at 25° C.

After that 200 spores were checked for signs of germination (evenly shaped spores, no change of size =no germination, spores showing a germination tube =germinated) and the percentage of germinated spores was calculated.

### EXAMPLE 3

### Increased Germination Rate of Dried Spores of Metarhizium as Compared to Non-Dried Spores

Spores of Metarhizium brunneum strain F52 were obtained using solid state fermentation. After separation from the substrate each fermentation batch was divided into fractions each of which was subjected to a vacuum drying procedure to dry to a specific residual moisture content. Spore batches obtained by using two different fermentation substrates, each based on cereals, with subsequent vacuum drying were packaged in sealed aluminum bags and stored until further use. The germination rate was determined according to the procedure described in Example 2.

As can be seen in FIGS. 1 and 2, Metarhizium spores dried to a residual moisture content of 2 wt.-%, but also of 4 wt.-% showed superior storage stability as compared to spores with higher residual moisture content, in particular a residual moisture content of 12% and above.

### EXAMPLE 4

### Increased Germination Rate of Dried Spores of Beauveria bassiana as Compared to Non-Dried Spores

Spores of Beauveria bassiana strain PPRI5339 were obtained using solid state fermentation. After separation from the substrate the spores was divided into fractions each of which was subjected to a vacuum drying procedure to dry to a specific residual moisture content. Spore samples were then stored at 25 or 30° C., respectively and samples taken at certain points in time.

As can be seen in Table 1 below, the germination rate of the spores did not significantly decrease at a residual moisture content of 4% at 25° C. as compared to a residual moisture content of 8%.

TABLE 1

| Residual | | Initial | Germination | Germination |
|---|---|---|---|---|
| moisture | Storage | germin- | rate | rate |
| content | temperature | ation rate | (3 months, | (6 months, |
| (%) | (° C.) | (%) | %) | %) |
| 4 | 25° C. | 87.6% | 86.6% | 88.8% |
| 4 | 30° C. | 87.6% | 80.7% | 74.4% |
| 8 | 25° C. | 85.3% | 67.9% | 54.4% |
| 8 | 30° C. | 85.3% | 53.3% | 33.4% |

### Example 5: Increased Germination Rate of Dried Spores of Isaria fumosorosea With Decreased Moisture Content

Spores of Isaria fumosorosea strain APOPKA97 were obtained using solid state fermentation. After separation from the substrate the spores were divided into fractions each of which was subjected to a vacuum drying procedure to dry to a specific residual moisture content. Spore samples were then stored at 25° C. and samples taken at certain points in time.

As can be seen in Table 2 below, the germination rate of the spores with the highest moisture level (10%) starts to decrease after 3 months as compared to the other fractions with lower moisture contents.

TABLE 2

| Residual | Storage | Initial | Germination | Germination |
|---|---|---|---|---|
| moisture | temperature | germination | rate [1 | rate [3 |
| content [%] | [° C.] | rate [%] | month, %] | months, %] |
| 2% | 25 | 100 | 98.77 | 99.5 |
| 3% | 25 | 99.5 | 99.8 | 99.51 |
| 4% | 25 | 99.5 | 99.77 | 97.77 |
| 6% | 25 | 99.5 | 99.04 | 97.07 |
| 10% | 25 | 100 | 97.31 | 93.4 |

1. A method of producing fungal spores with prolonged storage stability or of prolonging the storage stability of fungal spores comprising subjecting fungal spores to a drying procedure to result in a residual moisture content of less than 12%.

2. The method according to claim 1, wherein said residual moisture content is between 1.5 wt.-% and 6 wt.-%.

3. The method according to claim 1, wherein the germination rate of said spores after 6 months at room temperature is at least 80%.

4. The method according to claim 1, wherein said drying procedure comprises vacuum drying.

5. The method according to claim 4, wherein said vacuum drying is effected for up to 16 hours.

6. The method according to claim 4, wherein said vacuum drying is effected at a temperature of up to 30° C.

7. The method according to claim 4 wherein said vacuum drying is effected at a pressure of 10-1 mbar.

8. The method according to claim 1, wherein said fungal spores are conidia.

9. The method according to claim 1, wherein said fungal spores are from a fungus having beneficial activity in the area of plant protection.

10. The method according to claim 9, wherein said fungus has fungicidal, insecticidal, nematicidal or herbicidal activity or supports and/or promotes and/or stimulates plant health and/ or plant growth.

11. The method according to claim 9, wherein said fungus has fungicidal activity and is selected from the group consisting of Coniothyrium minitans, in particular strain CON/M/91-8 (Accession No. DSM-9660) (available as Contans ® from Prophyta, DE); Microsphaeropsis ochracea strain P130A (ATCC 74412); Aspergillus flavus, strain NRRL 21882 (available as Afla-Guard® from Syngenta) and strain AF36 (available as AF36 from Arizona Cotton Research and Protection Council, US); Gliocladium roseum, strain 321U from Adjuvants Plus; Talaromyces flavus, strain VII7b; Ampelomyces quisqualis, in particular strain AQ 10 (available as AQ 10® by IntrachemBio Italia); Gliocladium catenulatum (Synonym: Clonostachys rosea f. catenulate), in particular strain J1446 (e.g. Prestop® by Verdera Oy), strain IK726, strain 88-710 (WO2007/107000), strain CR7 (WO2015/035504), Trichoderma asperellum, in particular strain ICC 012 from Isagro or strain SKT-1 (e.g. ECO-HOPE® from Kumiai Chemical Industry), strain T34 (e.g. T34 Biocontrol by Biocontrol Technologies S.L., ES); Trichoderma viride, in particular strain TV1(e.g. Trianum-P by Koppert), strain B35 (Pietr et al., 1993, Zesz. Nauk. A R w Szczecinie 161: 125-137), Trichoderma atroviride, in particular strain CNCM 1-1237 (e.g. Esquive® WP from Agrauxine, FR), strain SC1 described in International Application No. PCT/IT2008/000196), strain 77B (T77 from Andermatt Biocontrol), strain no. V08/002387, strain NMI no. V08/002388, strain NMI no. V08/002389, strain NMI no. V08/002390, strain LC52 (e.g. Sentinel from Agrimm Technologies Limited), strain LUI32 (e.g. Tenet by Agrimm Technologies Limited), strain ATCC 20476 (IMI 206040), strain T11 (IM1352941/ CECT20498), strain SKT-1 (FERM P-16510), strain SKT-2 (FERM P-16511), strain SKT-3 (FERM P-17021) and Cladosporium cladosporioides, e.g. strain H39 (by Stichting Dienst Landbouwkundig Onderzoek).

12. The method according to claim 9, wherein said fungus has insecticidal activity and is selected from C2.3 Beauveria bassiana, in particular strain ATCC 74040; strain GHA (Accession No. ATCC74250); strain ATP02 (Accession No. DSM 24665); strain PPRI 5339; strain PPRI 7315, strain R444, strains IL197, IL12, IL236, IL10, IL131, IL116; strain BaGPK; strain ICPE 279, strain CG 716; C2.6 Lecanicillium lecanii (formerly known as Verticillium lecanii), in particular conidia of strain KV01, strain DAOM198499 or strain DAOM216596; C2.9 Lecanicillium muscarium (formerly Verticillium lecanii), in particular strain VE 6/CABI(=IMI) 268317/CBS102071/ARSEF5128; C2.10 Metarhizium acridum, e.g. ARSEF324 or isolate IMI 330189 (ARSEF7486); C2.11 Metarhizium brunneum, e.g. strain Cb 15 or strain F52 (DSM3884/ ATCC 90448); C2.12 Metarhizium anisopliae complex species/strains, e.g. strain ESALQ 1037, strain E-9, strain M206077, strain C4-B (NRRL 30905), strain ESC1, strain 15013-1 (NRRL 67073), strain 3213-1 (NRRL 67074), strain C20091, strain C20092, or strain ICIPE 78; C2.14 Paecilomyces fumosoroseus (new: Isaria fumosorosea), in particular strains Apopka 97, Fe9901, ARSEF 3581, ARSEF 3302, ARSEF 2679, IfB01 (China Center for Type Culture Collection CCTCC M2012400), ESALQ1296, ESALQ1364, ESALQ1409, CG1228, KCH J2, HIB-19, HIB-23, HIB-29, HIB-30, CHE-CNRCB 304, EH-511/3, CHE-CNRCB 303, CHE-CNRCB 305, CHE-CNRCB 307, EH-506/3, EH-503/3, EH-520/3, PFCAM, MBP, PSMB1, RCEF3304, PF01-N10 (CCTCC No. M207088), CCM 8367, SFP-198, K3, CLO 55, IfTS01, IfTS02, IfTS07, P1, If-02, If-2.3, If-03, Ifr AsC, PC-013 (DSMZ 26931), P43A, PCC, Pf04, Pf59, Pf109, FG340, Pfr1, Pfr8, Pfr9, Pfr10, Pfr11, Pfr12, Ifr531, IF-1106, I9602, I7284, I03011 (Patent U.S. Pat. No. 4,618,578), CNRCB1, SCAU-IFCF01, PF01-N4, Pfr-612, Pf-Tim, Pf-Tiz, Pf-Hal and Pf-Tic.

13. The method according to claim 9, wherein said fungus has nematicidal activity and is selected from D2.1 Muscodor albus, in particular strain QST 20799 (Accession No. NRRL 30547); D2.2 Muscodor roseus, in particular strain A3-5 (Accession No. NRRL 30548); D2.3 Paecilomyces lilacinus (also known as Purpureocillium lilacinum), in particular P. lilacinus strain 251 (AGAL 89/030550; e.g. BioAct from Bayer CropScience Biologics GmbH); D2.4 Trichoderma koningii; D2.5 Harposporium anguillullae; D2.6 Hirsutella minnesotensis; D2.7 Monacrosporium cionopagum; D2.8 Monacrosporium psychrophilum; D2.9 Myrothecium verrucaria, in particular strain AARC-0255 (e.g. DiTera™ by Valent Biosciences); D2.10 Paecilomyces variotii, strain Q-09 (e.g. Nemaquim® from Quimia, MX); D2.11 Stagonospora phaseoli (e.g. from Syngenta); D2.12 Trichoderma lignorum, in particular strain TL-0601 (e.g. Mycotric from Futureco Bioscience, ES); D2.13 Fusarium solani, strain Fs5; D2.14 Hirsutella rhossiliensis; D2.15 Monacrosporium drechsleri; D2.16 Monacrosporium gephyropagum; D2.17 Nematoctonus geogenius; D2.18 Nematoctonus leiosporus; D2.19 Neocosmospora vasinfecta; D2.20 Paraglomus sp, in particular Paraglomus brasilianum; D2.21 Pochonia chlamydosporia (also known as Vercillium chlamydosporium), in particular var. catenulata (IMI SD 187; e.g. KlamiC from The National Center of Animal and Plant Health (CENSA), CU); D2.22 Stagonospora heteroderae; D2.23 Meristacrum asterospermum, D2.24 Duddingtonia flagrans.

14. The method according to claim 9, wherein said fungus supports and/or promotes and/or stimulates plant health and plant growth and is E2.1 Talaromyces flavus, in particular strain V117b; E2.2 Trichoderma atroviride, in particular strain no. V08/002387, strain no. NMI No. V08/002388, strain no. NMI No. V08/002389, strain no. NMI No. V08/002390, strain LC52 (e.g. Sentinel from Agrimm Technologies Limited), strain kd (e.g. T-Gro from Andermatt Biocontrol), and/or strain LUI32 (e.g. Tenet from Agrimm Technologies Limited); E2.3 Trichoderma harzianum, in particular strain ITEM 908 or T-22 (e.g. Trianum-P from Koppert); E2.4 Myrothecium verrucaria, in particular strain AARC-0255 (e.g. DiTera™ from Valent Biosciences); E2.5 Penicillium bilaii, in particular strain ATCC 22348, and/or strain ATCC20851 (e.g. JumpStart® from Monsanto BioAg); E2.6 Pythium oligandrum, in particular strains DV74 or M1 (ATCC 38472; e.g. Polyversum from Bioprepraty, CZ); E2.7 Rhizopogon amylopogon (e.g. comprised in Myco-Sol from Helena Chemical Company); E2.8 Rhizopogon fulvigleba (e.g. comprised in Myco-Sol from Helena Chemical Company); E2.9 Trichoderma harzianum, in particular strain TSTh20, strain KD, product Eco-T from Plant Health Products, ZA or strain 1295-22; E2.10 Trichoderma koningii; E2.11 Glomus aggregatum; E2.12 Glomus clarum; E2.13 Glomus deserticola; E2.14 Glomus etunicatum; E2.15 Glomus intraradices; E2.16 Glomus monosporum; E2.17 Glomus mosseae; E2.18 Laccaria bicolor; E2.19 Rhizopogon luteolus; E2.20 Rhizopogon tinctorus; E2.21 Rhizopogon villosulus; E2.22 Scleroderma cepa; E2.23 Suillus granulatus; E2.24 Suillus punctatapies; E2.25 Trichoderma virens, in particular strain GL-21; and E2.26 Verticillium albo-atrum (formerly V. dahliae), in particular strain WCS850 (CBS 276.92; e.g. Dutch Trig from Tree Care Innovations).

15. The method of claim 1, wherein the fungal spores are from the species Metarhizium brunneum or Metarhizium acridum.

16. The method according to claim 15, wherein said fungal spores are from Metarhizium brunneun strain F52 or Metarhizium acridum strain ARSEF324.

17. The method according to claim 16, wherein the germination rate of said fungal spores after 6 months at room temperature is at least 85%.

18. The method according to claim 16, wherein the germination rate of said fungal spores after 8 months at room temperature is at least 80%.

19. The method of claim 16, wherein said drying is effected to a residual moisture content of between 1 and 5%.

20. The method of claim 1 wherein the fungal spores are from the species Isaria fumosorosea.

21. The method according to claim 20, wherein the fungal spores are from Isaria fumosorosea strain APOPKA97, Fe9901, ARSEF 3581, IfB01, ESALQ1296, ESALQ1364, ESALQ1409, CG1228, KCH J2, HIB-19, HIB-23, HIB-29, or HIB-30.

22. The method according to any one of claims 1 to 21, claim 1, further comprising packaging said spores under reduced oxygen and/or water exposure.

23. Fungal spores obtained by the method of claim 1.

24. A composition comprising the fungal spores according to claim 23.

25. A method of producing a composition, comprising formulating the fungal spores according to claim 23.

26. A method for controlling phytopathogenic fungi, insects, spiders, molluscs, weeds, rodents and/or nematodes in a plant, for enhancing growth of a plant or for increasing plant yield or root health, comprising applying fungal spores according to claim 23 to said plant or to a plot where plants are to be grown.

27. A method for controlling phytopathogenic fungi, insects, spiders, molluscs, weeds, rodents and/or nematodes in a plant, for enhancing growth of a plant or for increasing plant yield or root health, comprising applying a composition according to claim 24 to said plant or to a plot where plants are to be grown.
